(19) United States
(12) Patent Application Publication
(10) Pub. No.: US 2005/0276839 A1
(43) Pub. Date: Dec. 15, 2005

(54) Appetite satiation and hydration beverage

(76) Inventor: Calman H. Rifkin, Staten Island, NY (US)

Correspondence Address: STROOCK & STROOCK & LAVAN LLP, 180 MAIDEN LANE, NEW YORK, NY, 10038, US

(21) Appl. No.: 11/149,042

(22) Filed: Jun. 9, 2005

Related U.S. Application Data
(60) Provisional application No. 60/578,933, filed on Jun. 10, 2004.

(51) Int. Cl. A61K 35/78; A61K 47/00

(52) U.S. Cl. 424/439; 424/729; 424/725; 424/762; 424/641; 424/702; 424/722; 424/766; 424/771; 424/94.1; 514/59; 514/474; 514/574; 514/251; 514/276; 514/18; 514/456; 514/52

(57) ABSTRACT

Appetite satiation and hydrating beverages and methods of delivering appetite satiation ingredients and nutrients are disclosed. According to one embodiment of the invention, the appetite satiationhydrating beverage comprises at least one complex carbohydrate, at least one chelated electrolyte, hoodia gordonii cactus, gymnema sylvestre, betaine and piperine.

### CROSS REFERENCE TO RELATED APPLICATIONS

This application is claiming the benefit of U.S. Provisional Application Ser. No. 60/578,933, filed Jun. 10, 2004.

### BACKGROUND OF THE INVENTION

The invention relates generally to a food product and more particularly to a supplement that can promote a healthy lifestyle.

With the percentage of the population that is overweight, there has been a growing market for diet products. One of these types of products is appetite suppressants. A large number of these exist. Some are stimulants and others make the user nauseous. Rarely are they fully satisfactory for the typical user.

Although animal subjects need to lose weight or maintain a healthy weight, and properly nourish the body, animal subjects prefer to ingest substances, whether in solid or liquid form that taste good. Many subjects do not like to take pills. Subjects seeking to lose weight ingesting appetite suppressants will often malnourish their body because they are not hungry. Other subjects seeking to provide proper nutrition to their body, often over nourish their bodies with excess fats, proteins and sugars that cause unwanted weight gain. In addition many nutritional supplements lack efficient absorption mechanisms.

Accordingly, there is a need for an improved nutritional supplement that can promote a healthy lifestyle and avoid drawbacks of prior art.

### BRIEF SUMMARY OF THE INVENTION

Generally speaking in accordance with the invention, a food supplement product containing an appetite suppressant together with sufficient nutritional value to help prevent improper nutrition. One embodiment of the invention is directed to a beverage that can be administered to a subject to satiate appetite for the purpose of reducing weight by decreasing caloric intake and also hydrate the body, increase fat metabolism and physical energy, prevent dehydration and/or replace electrolytes, nutrients and minerals that are lost during periods of activity or stress.

One embodiment of the invention is directed to a composition for appetite satiation and hydration comprising at least one complex carbohydrate, at least one chelated electrolyte, and one or more of Hoodia Gordonii Cactus Extract, Hoodia Gordonii Cactus in the form of a concentrated powder, a puree or a juice, Gymnema Sylvestre, Betaine, and Piperine in effective amounts to accomplish appetite satiation and hydration.

Another embodiment of the invention can be a composition for appetite satiation and hydration comprising at least one complex carbohydrate, at least one chelated electrolyte, at least one of Hoodia Gordonii Cactus Extract, Gymnema Sylvestre, Hydroxycitrate, Green Tea Leaf Extract, Green Tea Leaf in the form of a powder, Betaine, and Piperine in effective amounts to accomplish appetite satiation and hydration.

The invention can comprise a beverage provided in an easy to open container in the amount from about 200 ml to 1000 ml. This should contain at least one of, and preferably all of: Hoodia Gordonii Cactus from about 50 mg/L to about 500 mg/L, more preferably from about 200 mg/L to about 400 mg/L, and most preferably from about 275 mg/L to about 350 mg/L, Gymnema Sylvestre from about 50 mg/L to about 500 mg/L, more preferably from about 80 mg/L to about 400 mg/L, and most preferably from about 275 mg/L to about 350 mg/L, Green Tea Leaf Extract from about 50 mg/L to about 1000 mg/L, more preferably from about 100 mg/L to about 900 mg/L, and most preferably from about 300 mg/L to about 400 mg/L, Hydroxcitrate from about 50 mg/L to about 500 mg/L, more preferably from about 80 mg/L to about 400 mg/L, and most preferably from about 275 mg/L to about 350 mg/L, Betaine from about 5 mg/L to about 125 mg/L, more preferably from about 15 mg/L to about 100 mg/L, most preferably from about 35 mg/L to 65 mg/L, and Piperine from about 1.1 mg/L to about 50 mg/L, more preferably from about 2 mg/L to about 20 mg/L, most preferably from about 3 mg/L to 10 mg/L.

An embodiment of the invention can also contain chelated electrolytes, including but not limited to the aspartate forms of potassium and magnesium. An embodiment of the invention may also contain antioxidants, including but not limited to vitamin Ester-C, vitamin E, selenium, coenzyme Q 10, pycnogenol, L-glutathione, superoxide dismutase, bioflavonoids, pine bark grape seed complex, selenium, zinc, garlic or combinations thereof. An embodiment of the invention can contain sodium compounds, including but not limited to one or more of sodium chloride, sodium citrate, sodium benzoate or combinations thereof.

An embodiment of the invention can also contain a sweetening agent, including but not limited to one or more of maltodextrin, neohesperidine dihydrochalcone, Lo Han Fruit extract, sucralose, or combinations thereof. An embodiment of the invention can contain herbal extracts, including but not limited to Curcumin, Boswellin, Ashwagandha, Ginkgo Biloba, Capsaicin, Aconitine or combinations thereof.

Embodiments of this invention can be carbonated or in concentrate syrup or power form. Embodiments of this invention can further contain vitamins, including but not limited tovitamin B1, vitamin B2, niacinamide, vitamin B6, vitamin B12, folic acid, vitamin C, vitamin E or combinations thereof, minerals, including but not limited to calcium, zinc, vanadium, selenium, chromium, boron, potassium, manganese, copper, magnesium or combinations thereof, amino acids, including but not limited to, L-carnitine, lysine, isoleucine, leucine, threonine, valine, tryptophan, phenylalanine, methionine, L-selenomethionine, DL-phenylalanine, tyrosine or combinations thereof.

Further embodiments may contatin other ingredients including but not limited to, malic acid, citric acid, herbs, natural flavors, natural colors, preservatives or combinations thereof.

### DESCRIPTION OF THE PREFERRED EMBODIMENTS

This invention relates to a nutraceutical beverage that can increase fat metabolism and physical energy, as well as satiate appetite for the purpose of reducing weight by decreasing caloric intake and hydrate the body. The term nutraceutical beverage as used herein includes an appetite satiation beverage, an appetite satiation and hydrating beverage, diet nutraceutical beverage, a low calorie nutraceutical beverage, rehydration beverage, a rehydration fitness or sports beverage, an animal rehydration beverage, and a pediatric nutraceutical beverage. For the purpose of this invention the term to satiate will also mean to satisfy, make content, gratify, curb, and/or suppress a subject's appetite.

Some appetite satiation compositions including but not limited to diet pills may cause a subject to be malnourished as a result lack of nutrients, electrolytes, minerals and hydration due to lack of appetite. Some nutritive drinks, including but not limited to protein or amino acid drinks, sports drinks or vitamin water drinks sweetened with sugar may cause a subject to be overly nourished and cause the subject an unwanted weight increase. The present invention can provide appetite satiation and nutrition, thus avoiding the unwanted effects of malnourishment, or unwanted weight increase to over-nourishment.

The present invention relates to a beverage composition that can be administered to a subject to satiate appetite, prevent dehydration and/or replace electrolytes, nutrients and minerals that are lost during periods of activity or stress. The present invention relates to appetite satiation beverages and a method for enhancing hydration; preferably with enhanced absorption after ingestion while at the same time attenuating muscle fatigue and, preferably, destroying harmful free radicals. The present invention is applicable to the rehydration of animals, including but not limited to, human beings at rest, before meals, and a dehydrating event, including but not limited to exercise. The present invention is applicable to domestic and/or farm animals, including by not limited to dogs and cats, at rest, as part of a diet, or after a dehydrating event. The present invention has pediatric applications as a composition of the invention has high nutritive value without excess calories and therefore may be healthier than normal fruit juices for certain pediatric subjects. The present invention also provides a method for replacing water and carbohydrates lost by perspiration and for preventing a decrease in the glucose content of blood during periods of heavy muscle work. The invention also comprises a process for preparing the beverage product.

The present invention relates to a new liquid composition for a nutraceutical drink that satiates appetite, increases fat metabolism and sensitizes the “glucostat” in the brain that monitors blood sugar availability. This invention provides overall hydration and replenishes body electrolytes (Sodium, Potassium, Calcium, Magnesium) lost due to physical exertion and stress. More precisely, the present invention relates to carbonated or non-carbonated beverage compositions and beverage concentrates adapted for oral administration of water, physiologically essential electrolytes and other ingredients to a subject specifically adapted for appetite satiation and enhancing hydration; preferably with enhanced absorption after ingestion while at the same time attenuating muscle fatigue and, preferably, destroying harmful free radicals.

A composition relating to the present invention can provide improved digestion of carbohydrates through the use of an amino acid chelate, including but not limited to boron, selenium, manganese and molybdenum, to stimulate and enhance enzymatic carbohydrase activity. Optionally, sweetening agents, antioxidants to destroy free radicals generated during exercise, and certain amino acids that supply protein for the recovery period can also be included in the instant compositions relating to this invention.

The beverages and methods of the instant invention are ideally suited to anyone who desires appetite satiation, has been exposed to above-normal heat or hydration stress, such as those living in hot or humid climates, factory workers, armed forces personnel, police, firemen, airline workers & passengers and the like, in addition to those who engage in exercise.

The nutraceutical beverage contains appetite satiation ingredients, including, but not limited to, Hoodia Gordonii Cactus, Green Tea Leaf Extract, Hydroxycitrate, L-Carnitine, Gymnema Sylvestre, Tyrosine and Coenzyme Q10; fitness ingredients including, but not limited to, Chromium Picolinate and Chromium Picolinate; minerals and vitamins including but not limited to Calcium, Magnesium, Potassium, Zinc, Pyridoxine Hydrochloride (Vitamin B6), Cyanocobalamin (Vitamin B12), Folic Acid, Niacin and Calcium Pantothenate. Furthermore, the beverage may be stabilized with Malic acid to reduce the pH below 4.6 for hot fill or without Malic acid for aseptic processing. The nutraceutical beverage may also contain non-nutritive sweeteners, masking flavor, fruit flavors, and food coloring. Water prepared from such ingredients may have a pH in the range of about 4.0-7.0 and may be processed at about 195-210 F. or aseptically. The advantage of the nutraceutical beverage is satiation of appetite, increased overall physical energy including, but not limited to, increased performance and endurance, appetite suppression and inhibition of fat production to help weight loss. Furthermore, the beverage provides essential minerals and vitamins thereby enhancing the intake of fluids.

The energy ingredients, L-Carnitine, Coenzyme Q10, Green Tea Leaf Extract, and Tyrosine may be optionally included in the nutraceutical drink to provide an alternative hydration beverage to consumers and provide energy. The lack of carbonation and added minerals, vitamins, and amino acids, permit a subject to more rapidly ingest water during periods of thirst. The inclusion of flavors without added calories provides additional taste without the concern of excess calories.

Ingredients of exemplary compositions are now described in further detail below.

Water: Although any type of filtered water or tap water may be used effectively, Reverse osmosis, de-ionized, chlorine free, filtered water is important for this nutraceutical drink because the mineral content of other type of filtered or tap water may adversely effect the taste.

Appetite Satiation Ingredients:

Hoodia Gordonii Cactus: Hoodia Gordonii Cactus contains a molecule that is estimated to be up to 100,000 times as potent as glucose in sending a signal to the brain that the body is in a state of satiety, or in common terms not hungry.

Hydroxycitrate: The effects of hydroxycitrate are based on its action as a potent inhibitor of the enzyme ATP (Adenosine Triphosphate) citrate lyase (also known as citrate cleavage enzyme), which is required for the synthesis of fatty acids. This enzyme takes citrate, which has been exported from the mitochondria to the cytoplasm, and creates acetyl CoA and oxaloacetate from it.

L-Carnitine: Research indicates that L-Carnitine enhances aerobic performance and allows the body to exercise longer without fatigue. L-Carnitine also aids cardiac function by fueling the heart with energy, raises HDL levels, lowers triglycerides and reduces hypertension. It also assists in the burning of body fat.

Coenzyme Q10: Coenzyme Q10 is a powerful antioxidant. It plays a critical role in the production of energy in every cell of the body. Coenzyme Q10 aids in metabolism, circulation, and stimulates the immune system.

Gymnema Sylvestre: Gymnema Sylvestre (GS) is a woody climbing plant that grows in the tropical forests of central and southern India. The leaves are used in herbal medicine preparations. The leaves, when chewed, interfere with the ability to taste sweetness. Gymnema Sylvestre has been previously used as a stomachic, diuretic, refrigerant, astringent, and tonic. It has been found to increase urine output and reduce hyperglycemia in both animal and human studies.

Tyrosine: Tyrosine is a nonessential amino acid that is synthesized in the body from phenylalanine. As a building block for several important brain chemicals, tyrosine is needed to make epinephrine, norepinephrine, serotonin, and dopamine, all of which work to regulate mood. Deficiencies in tyrosine, therefore, have been associated with depression. Tyrosine also aids in the production of melanin (pigment responsible for hair and skin color) and in the function of organs in the body responsible for making and regulating hormones, including the adrenal, thyroid, and pituitary glands. Tyrosine is also involved in the synthesis of enkephalins, substances that have pain-relieving effects in the body. Low levels of tyrosine have been associated with low blood pressure, low body temperature, and an under active thyroid.

Green Tea Leaf Extract: Green Tea Leaf Extract works by inhibiting the enzyme, catechol O-methyltransferase (COMT), which is responsible for breaking down noreinephrine, thus increasing this neurotransmitter's life in the synaptic cleft. Also by inhibiting phosphodiesterases it increases the life of cyclic AMP (adenosine monophosphate) (cAMP) in the cell. Together, these effects heighten the impact of norepinephrine is the neurotransmitter which plays a large role in the control of thermogenesis and fat oxidation for weight loss.

Nutrition Ingredients:

Chromium Picolinate: Chromium Picolinate and Diachrome are an exceptional bioactive source of the essential mineral chromium. Chromium plays a vital role in “sensitizing” the body's tissues to the hormone insulin. Research indicates that chromium picolinate helps to control appetite especially sugar cravings. It is believed that chromium picolinate sensitizes the “glucostat” in the brain that monitors blood sugar availability and tells you when you are hungry or not hungry. Chromium picolinate also helps to preserve muscle in dieters so that they burn more fat and less muscle. Clinical trials with 200 to 400 micrograms of Chromium picolinate daily produced significant benefits. (Gilbert R. Kaats, Kenneth Blum, Dennis Pullin, Samuel C. Keith and Robert Wood. “A randomized, double-masked, placebo-controlled study of the effects of chromium picolinate supplementation on body composition: a replication and extension of a previous study,” Current Therapeutic Research 59(6):379-388, 1998. Gilbert R. Kaats, Kenneth Blum, Jeffrey A. Fisher and Jack A. Adelman. “Effects of chromium picolinate supplementation on body composition: a randomized, double-masked, placebo-controlled study,” Current Therapeutic Research 57(10):747-456, 1996.) Each of these references are incorporated by reference.

Piperine: Piperine, or mixtures containing piperine, have been shown to increase the bioavailability, blood levels and efficacy of a number of drugs including ingredients of vasaka leaves (Bose, K. G., (1928) Pharmacopeia India, Bose Laboratories, Calcutta), vasicine (Atal et al., Journal of Ethnopharmacology, 4, 229-233 (1981)), sparteine (Atal et al., ibid), sulfadiazine (Atal et al., ibid), rifampicin (Zutshi, U. et al. (1984) Journal of the Association of Physicians of India, 33, 223-224), phenytoin (Bano et al., Planta Medica, 1987, pp. 568-569), pentobarbitone (Majumdar, A. N. et al. (1990), Indian Journal of Experimental Biology, 28, 486-487), theophylline (Bano et al., Eur. J. Clin. Pharmacol. (1991) 41:615-617) and propranolol (ibid). Each of these references are incorporated by reference.

Betaine: Betaine is a metabolite of choline and is also known as Trimethylglycine or TMG. Betaine works by donating methyl groups. Many important biochemical processes (for example, proper liver function, cellular replication, and detoxification reactions) rely on methylation, therefore, supplementation has interesting potential health benefits. Betaine is a nutrient that plays a role in the health of the cardiovascular system. Betaine, along with other nutrients, helps to reduce potentially toxic levels of homocysteine (Hcy). A naturally occurring amino acid that can be harmful to blood vessels thereby contributing to the development of heart disease, stroke, and peripheral vascular disease. Betaine functions closely with other nutrients to break down homocysteine. Betaine also increases the concentration of acids in the stomach. Additionally, betaine has been found to reduce skin-irritating effects of cosmetics such as sodium lauryl sulfate. Betaine containing toothpaste has been shown to relieve the symptoms of dry mouth. (Soderling, E., et al., “Betaine-containing toothpaste relieves subjective symptoms of dry mouth,” Acta Odontol Scand, 1998; 56:65-9) Dietary sources of betaine include beets, fish, legumes, broccoli, and spinach. Each of foregoin references are incorporated by reference.

Minerals: Organic or inorganic salts of calcium, magnesium, zinc, and potassium or a premix of the aforementioned provide essential minerals for healthy joints, muscles, nerve functioning and help regulate blood pressure.

Vitamins: Water soluble Thiamin, Riboflavin, Pyridoxine hydrochloride (Vitamin B6), Cyanocobalamin (Vitamin B12), Folic Acid, Niacin, Biotin, Inositol, and PABA are B-complex vitamins and are added to compensate for B-vitamin deficiencies and also to optimize the metabolic pathways where B-complex vitamins are used as co-enzymes. The B-vitamin group is crucial for producing energy in the mitochondria of cells in the body.

Sweeteners: In some cases a non-nutritive or nutritive sweetener may be added to improve the taste and to encourage the drink-ability of the beverage without adding too many calories. Although simple sugars such as fructose or sucrose can be used, more preferred is a sweetening agent that comprises a complex carbohydrate or sweetening material conventionally used in beverages and in their usual concentrations. Examples include, but are not limited to, maltodextrin, neohesperidine dihydrochalcone, Lo Han Fruit extract, mixtures thereof, and the like. Other sweetening agents such as sucralose may also be used. In addition a higher calorie sweetner such as high fructose corn syrup may also be utilized and could be more applicable to a beverage designed for a use where limiting calories is not necessary to the application. For dietetic beverages the known artificial sweeteners, including, but not limited to Acesulfame potassium, can be substituted for the sweetening agents. Since many subjects who exercise do not want to ingest excess calories, the level of sugars used is kept low, for example about 2 to 4% by weight of the beverage. However, there are often exercise related needs for the ingestion of greater levels of carbohydrates, especially in the recovery from long duration exercise where carbohydrate (glucose) is needed to replace muscle glycogen.

Flavors: Water soluble natural flavors would provide for thirst quenching appeal and variety to the consumer.

Herbal Extracts: Extracts from herbs, including but not limited to Curcumin, Boswellin, Ashwagandha, Ginkgo Biloba, Capsaicin, Aconitine may have the effect of further enriching the beverage and be consistent with the goals and functions of the invention.

Potassium addresses the issue of attenuation of muscle fatigue due to exercise and prevention of a post-exercise rapid fall in blood potassium levels. An exemplary composition may contain potassium in an amount between about 4 to 4.5 mEq/L; however potassium levels as high as 6 mEq/L or greater can be used. Such higher levels may be contraindicated with certain individuals who have certain kidney and adrenal gland disorders, hypertension, and certain heart disorders such as frequently occurring abnormal heart rhythms. Suitable sources of potassium include, but are not limited to, edible salts such as chloride, sorbate, citrate, and the like and mixtures thereof. Potassium aspartate may also be utilized as discussed in detail below.

Certain edible water-soluble chelated electrolytes improve results before, during, and after exercise from the dehydrating and muscle fatigue problems of exercise. The advantage of these salts is that the chelate aspartate thereof becomes available promptly upon ingestion of the beverage. This results in a higher absorption of potassium into the blood stream allowing for improved hydration and an enhanced recovery period. An additional advantage of using the aspartate form is that it provides phosphate ions, which are beneficial in rehydration, as well as supporting muscle metabolism during exercise.

The compositions of the present invention may contain chelated electrolytes. Examples of suitable chelated electrolytes include, but are not limited to, potassium aspartate and magnesium aspartate. When these chelated aspartates are used, the amounts utilized are those that will supply a portion of the alkali metal, preferably potassium, within the ranges set forth above (a blood plasma level between 4 to 4.5 mEq/L and excretion of potassium in the urine).

In order to enhance the flow of saliva, an acidulent is employed in the beverage. Suitable acidulents include, but are not limited to, betaine hydrochloride, betaine citrate, tartaric acid, citric acid, fumaric acid, malic acid, ascorbic acid, phosphoric acid and mixtures thereof. An exemplary composition of this invention would contain betaine.

The composition of the present invention may also include piperine. Suitable forms of piperine include, but are not limited to, synthetically made piperine extract from black pepper, extract from piper longum, and Bioperine®. Bioperine® is commercially available from Sabinsa Corporation located in Piscataway, N.J.

Another result of exercise is the greatly increased production of destructive free radicals. To negate such harmful effects the nutraceutical beverages of the present invention may include at least one edible antioxidant in the composition which will react readily with the free radicals and destroy them before they can damage the body. Suitable antioxidants include, but are not limited to, Vitamin C, Vitamin E, selenium, Coenzyme Q-10, pycnogenol, L-glutathione, superoxide dismutase, bioflavonoids, pine bark grape seed complex, selenium, zinc, and antioxidants found in plants such as garlic. Of these, Vitamin C has the additional benefit of acting as a buffer in the beverages, thereby combating acidosis. A well known example of Vitamin C is Ester-C®. The amounts of antioxidant used can vary widely, but care must be taken to ensure that the amount of antioxidant used does not adversely affect the taste of the beverage.

Sodium can be used at levels conventionally utilized in rehydrating fluids and beverages, i.e., about 20 mEq/L and higher; although the higher levels may cause the beverage to taste too “salty” to the consumer. A preferred level is about 15 to 20 mEq/L. Any of the sodium salts commonly used in rehydrating fluids may be used. Suitable examples include, but are not limited to, sodium chloride, sodium citrate, sodium benzoate, other sodium salts and mixtures thereof.

Compositions of the present invention may also include vitamins, minerals and/or amino acids. Suitable vitamins include, but are not limited to, vitamin B1, vitamin B2, niacinamide, vitamin B6, vitamin B12, folic acid, vitamin C, and vitamin E. Suitable minerals include, but are not limited to calcium, iron, zinc, vanadium, selenium, chromium, boron, potassium, manganese, copper and magnesium. Suitable amino acids include, but are not limited to, lysine, isoleucine, leucine, threonine, valine, tryptophan, phenylalanine, methionine and L-selenomethionine,

It will be evident that the amount of water, or liquid, added is that required to provide the proper concentration of the active components for the size of the container for the beverage, normally, such beverages, as with other drinks, come in a container holding at least about 8 fluid ounces of the beverage.

A nutraceutical beverage according to the present invention comprises at least one complex carbohydrate, at least one chelated electrolyte, Hoodia Gordonii Cactus, Gymnema Sylvestre, Green Tea Leaf Extract, Hydroxycitrate, Betaine and Piperine.

Another exemplary composition of a nutraceutical beverage according to the present invention comprises at least one complex carbohydrate, at least one chelated electrolyte, Hoodia Gordonii Cactus in the range of about 80-400 mg/l, Gymnema Sylvestre in the range of about 80-400 mg/l, Green Tea Leaf Extract in the range of about 100-900 mg/l, Hydroxycitrate in the range of about 80-400 mg/l, Betaine in the range of about 15-100 mg/L and Piperine in the range of about 1.1-50 mg/L.

Another exemplary composition of a nutraceutical beverage according to the present invention would have a pH range from about 3.5 to 7.0 depending upon the packaging and processing used for the water. This exemplary nutraceutical beverage comprises at least one complex carbohydrate, at least one chelated electrolyte, Hoodia Gordonii Cactus, Gymnema Sylvestre, Green Tea Leaf Extract, Hydroxycitrate, Betaine and Piperine.

Furthermore, depending on the desired purpose of the nutraceutical beverage, any of the nutraceutical beverage described above, may include one or more of additional ingredients including but not limited to, Sodium Chloride, Potassium Aspartate, Vitamin C, Calcium Citrate, Magnesium Aspartate, Thiamin, Riboflavin, Niacin, Pyridoxine HCL (Vitamin B-6), Folic acid, Cyanocobalamin(Vitamin B-12), Choline Dihydrogen Citrate, Malic acid, α-Tocopherol acetate, g-Tocopherol, Inositol, PABA, Biotin, Zinc monomethionine aspartate, Cooper Sebacate, Manganese (as amino acid chelate), Molybdenum (as amino acid chelate), Vanadium Chelavite, Selenium (as amino acid chelate), Chromium Picolinate, Boron (as amino acid chelate), L-carnitine, DL-Phenylalanine, Tyrosine, Coenzyme Q10, OPC (95%) (Oligomeric Proanthocyanidin), Calcium Pantothenate, Sucralose, Acesulfame Potassium and Maltodextrin. Exemplary amounts of such additional ingredients are described in detail in Table 1.

TABLE 1

| | | |
|---|---|---|
| Sodium Chloride | 40-400 | mg/L |
| Potassium Aspartate | 100-500 | mg/L |
| Vitamin C | 30-400 | mg/L |
| Calcium Citrate | 50-400 | mg/L |
| Magnesium Aspartate | 20-200 | mg/L |
| Thiamin | 0.2-4 | mg/L |
| Riboflavin | 0.5-6 | mg/L |
| Niacin | 0.5-8 | mg/L |
| Pyridoxine HCL (Vitamin B-6) | 0.4-7 | mg/L |
| Folic acid | 40-400 | mcg/L |
| Cyanocobalamin (Vitamin B-12) | 1-20 | mcg/L |
| Choline Dihydrogen Citrate | 20-500 | mg/L |
| Malic acid | 25-250 | mg/L |
| α-Tocopherol acetate | 2-50 | IU/L |
| g-Tocopherol | 0.75-50 | IU/L |
| Inositol | 1-10 | mg/L |
| PABA | .5-2 | mg/L |
| Biotin | 25-250 | mcg/L |
| Zinc monomethionine aspartate | 10-100 | mcg/L |
| Cooper Sebacate | 10-100 | mcg/L |
| Manganese (as amino acid chelate) | 100-600 | mg/L |
| Molybdenum (as amino acid chelate) | 2-30 | mcg/L |
| Vanadium Chelavite | 2.75-30 | mcg/L |
| Selenium (as amino acid chelate) | 5-50 | mcg/L |
| Chromium Picolinate | 5-120 | mcg/L |
| Boron (as amino acid chelate) | 2-50 | mg/L |
| L-carnitine | 100-900 | mg/L |
| DL-Phenylalanine | 10-200 | mg/L |
| Tyrosine | 10-200 | mg/L |
| Coenzyme Q10 | 1-100 | mg/L |
| OPC (95%) | 2-100 | mg/L |
| Calcium Pantothenate | 1-10 | mg/L |
| Sucralose | 10-250 | mg/L |
| Acesulfame potassium | 20-80 | mg/L |
| Maltodextrin | 2-8 | g/l |

It is well within the purview of the skilled artisan to determine the amount of such additional ingredients based on the desired product. For example, a skilled artisan would choose to include relatively high amounts of Thiamin and Riboflavin if the resulting nutraceutical beverage was created for increased energy while dieting and having a decreased caloric intake.

The exemplary compositions of the present invention may take a variety of forms. For instance, the composition may be manufactured and sold as a ready-to-drink beverage in an easy to open container 200 to 1000 ml in size, for direct consumption by a subject. The exemplary compositions may be prepared in concentrate, syrup or powder form to be reconstituted for use by the subject by the addition of water or any other appropriate liquid. Such reconstitution is made with the requisite amounts of water/liquid to ensure that the beverage to be consumed contains the active components in the proportions previously noted. In another embodiment the composition may be solubilized in water/liquid and then brought to a frozen state, so as to provide—for example—flavored ices on sticks, like the ones known under the commercial name or trade mark “Popsicle.”

Furthermore, the exemplary compositions may be prepared by simply admixing the ingredients and packaging them in the conventional aseptic apparatus used for such purposes. Additionally, the solution, made up in reverse osmosis filtered water, may be combined with a small amount of a non glucose carbohydrate, such as, but not limited to, fructose or sorbitol to give it a pleasant sweet taste, with a calorie load not exceeding 120 kcal/liter, and flavored with any choice of fruit extract and/or aroma, such as orange, lemon, strawberry or others in any amount that yields a desirable taste to the end user. Sucralose, acesulfame potassium or high fructose corn syrup may also be added to give it a sweet taste in any amount that yields a desirable taste to the end user. The resulting beverage is preserved by pasteurization or sterilization and is intended for an average intake in conditions of environmental heat in amounts up to 2 liters/day.

In addition, the exemplary compositions may be prepared as a dry powder mixture. The dry powder mixture is combined with a support for dispersion in water/liquid, such as maltodextrin, with a non-glucose sweet taste base, such as neohesperidine dihydrochalcone, at a level so as not to exceed 120 kcal/L, when reconstituted, and possibly flavored with fruit extracts or aromas, such as orange, lemon, strawberry, or others. One dose is intended for dissolution in 591 ml drinking water/liquid (one standard sports bottle). The concentrations are merely indicative and more concentrated drinks may be prepared on the same or a similar formula basis.

The above powder mix may also be added to a suitable support for tablet compression, with good organoleptic properties, such as sorbitol and magnesium stearate. The mass is possibly edulcorated with a known natural sweetener such as Neohesperidine dihydrochalcone and the total energy content should not exceed 120 kcal in the average daily intake. The product may be flavored with any choice of fruit or other flavors, such as orange, lemon, menthol, eucaplytol, or the like. The compressed tablets (or equivalent solid forms with the same composition) are intended for an average daily intake of between 5 and 10 tablets.

### Other Examples

Although the prior exemplary compositions have been described in terms of compositional ranges, the skilled artisan will appreciate that the various substitutions, modifications, changes, and omissions may be made without departing from the spirit of the present invention.

The following examples are provided for illustration only and are not intended to be construed as limiting.

### Example 1

A lemon/lime or other natural fruit flavored nutraceutical beverage containing, for each 11 fluid ounces, the following:

| 1 WT. | | |
|---|---|---|
| Maltodextrin | 5 | grams |
| Sodium | 27 | milligrams |
| Potassium | 121 | milligrams |
| Ester-C ® (Vitamin C) | 55 | milligrams |
| Gymnema Sylvestre | 100 | milligrams |
| Betaine | 16.5 | milligrams |
| Piperine | 1.1 | milligrams |
| Hoodia Gordonii | 100 | milligrams |
| Green Tea Extract | 117.5 | milligrams |
| Hydroxycitrate | 100 | milligrams |

| | |
|---|---|
| Water and Other | Balance |
| Optional Ingredients | |

The sodium is added in the form of a mixture of citrate, benzoate, and chloride salts and the potassium in the form of citrate and sorbate salts. This example beverage also contains citric acid, natural flavors, and natural colors for their usual purpose and in the amount sufficient to give the beverage a lemon/lime-like or other natural fruit flavor and appearance. Preferably, the potassium and all of the other minerals besides sodium are chelated.

The nutraceutical beverage is prepared by simply admixing the ingredients and packaging them in the conventional aseptic apparatus used for such purposes.

### Example 2

A tea/lemon or other natural fruit flavored nutraceutical beverage containing, for each 11 fluid ounces, the following:

| 1 WT. | | |
|---|---|---|
| Maltodextrin | 5 | grams |
| Sodium | 27 | milligrams |
| Potassium | 121 | milligrams |
| Ester-C ® (Vitamin C) | 55 | milligrams |
| Gymnema Sylvestre | 100 | milligrams |
| Betaine | 16.5 | milligrams |
| Piperine | 1.1 | milligrams |
| Hoodia Gordonii | 100 | milligrams |
| Green Tea Extract | 117.5 | milligrams |
| Hydroxycitrate | 100 | milligrams |

| | |
|---|---|
| Water and Other | Balance |
| Optional Ingredients | |

The sodium is added in the form of a mixture of citrate, benzoate, and chloride salts and the potassium in the form of citrate and sorbate salts. This example beverage also contains citric acid, natural flavors, and natural colors for their usual purpose and in the amount sufficient to give the beverage a tea/lemon or other natural fruit flavor and appearance. Preferably, the potassium and all of the other minerals besides sodium are chelated.

The nutraceutical beverage is prepared by simply admixing the ingredients and packaging them in the conventional aseptic apparatus used for such purposes.

### Example 3

Hoodia Gordonii Cactus 300 mg/liter
L-Camitine 500 mg/liter
Gymnema Sylvestre 300 mg/liter
Green Tea Leaf Extract 350 mg/liter
Hydroxycitrate 300 mg/liter
Chromium Picolinate 100 mcg/liter
Calcium Citrate 70 mg/liter
Potassium Asparate 100 mg/liter
Magnesium Asparate 25 mg/liter
Zinc Monomethionine Asparate 10 mcg/liter
Selenium 5 mcg/liter
Vitamin B6 0.7 mg/liter
Vitamin B12 2.12 mcg/liter
Folic Acid 140 mcg/liter
Niacin 2.75 mg/liter
Calcium Pantothenate 1.5 mg/liter
Vitamin C 43 mg/liter
Natural aroma 150 mg/liter
Water balance

The foregoing composition contains no sweetener and the pH is 4.3. The purpose of this nutraceutical beverage is to provide ingredients that are known and scientifically studied to provide appetite satiation, energy, fitness to lose weight, replenish fluids, vitamins, and electrolytes without altering the taste of the nutraceutical beverage. The appetite satiation hydrating drink is typically ingested at intervals prior to meals.

### Example 4

Hoodia Gordonii Cactus 300 mg/liter
Green Tea Leaf Extract 350 mg/liter
Gymnema Sylvestre 300 mg/liter
Hydroxycitrate 300 mg/liter
Chromium Picolinate 100 mcg/liter
Calcium Citrate 70 mg/liter
Potassium Asparate 100 mg/liter
Magnesium Asparate 25 mg/liter
Zinc Monomethionine Asparate 10 mcg/liter
Selenium 5 mcg/liter
Vitamin B6 0.7 mg/liter
Vitamin B12 2.12 mcg/liter
Folic Acid 140 mcg/liter
Ester-C® (Vitamin C) 43 mg/liter
Sucralose 13.0 mg/liter
Natural aroma 150 mg/liter
Water balance

The foregoing nutraceutical beverage has a pH of 4.5, and contains only non-nutritive sweetener.

### Example 5

Hoodia Gordonii Cactus 300 mg/liter
Gymnema Sylvestre 300 mg/liter
Green Tea Leaf Extract 350 mg/liter
Hydroxycitrate 300 mg/liter
Chromium Picolinate 100 mcg/liter
Calcium Citrate 70 mg/liter
Potassium Asparate 100 mg/liter
Magnesium Asparate 25 mg/liter
Zinc Monomethionine Asparate 10 mcg/liter
Selenium 5 mcg/liter
Vitamin B6 0.7 mg/liter
Vitamin B12 2.12 mcg/liter
Folic Acid 140 mcg/liter
Ester-C® (Vitamin C) 43 mg/liter
Sucralose 250 mg/liter
Natural aroma 150 mg/liter
Water balance

The nutraceutical beverage has the pH 5.8.

### Example 6

Hoodia Gordonii Cactus 300 mg/liter
Green Tea Leaf Extract 350 mg/liter
Gymnema Sylvestre 300 mg/liter
Hydroxycitrate 300 mg/liter
Chromium Picolinate 100 mcg/liter
Calcium Citrate 70 mg/liter
Potassium Asparate 100 mg/liter
Magnesium Asparate 25 mg/liter
Zinc Monomethionine Asparate 10 mcg/liter
Selenium 5 mcg/liter
Vitamin B6 0.7 mg/liter
Vitamin B12 2.12 mcg/liter
Folic Acid 140 mcg/liter
Niacin 2.75 mg/liter
Calcium Pantothenate 1.5 mg/liter
Ester-C® (Vitamin C) 43 mg/liter
Sucralose 62 mg/liter
Acesulfame potassium 60 mg/liter
Citric Acid 1000 mg/liter
Natural aroma 150 mg/liter
Natural Berry Flavor 300 mg/liter
Water balance

### Example #7

Hoodia Gordonii Cactus 100 mg/liter
Green Tea Leaf Extract 100 mg/liter
Gymnema Sylvestre 100 mg/liter
Hydroxycitrate 100 mg/liter
Piperine 1.1 mg/liter
Betaine Citrate 16.5 mg/liter
Vitamin C (from Ester C®) 55 mg/liter
Thiamin (as Thiamin HCl) 0.41 mg/liter
Riboflavin 0.74 mg/liter
Niacin (as Niacinamide) 1.38 mg/liter
Pyridoxine HCl (Vitamin B6) 0.55 mg/liter
Folic Acid 55 mcg/liter
Calcium Pantothenate 2.5 mg/liter
Cyanocobalamin (Vitamin B12 ) 1.65 mcg/liter
α-Tocopherol Acetate 2 IU/liter
g-Tocopherol 0.75 IU/liter
Biotin 27.5 mcg/liter
Calcium Citrate 55 mg/liter
Malic Acid 27.5 mg/liter
Boron (as amino-acid chelate) 2.75 mg/liter
Phosphorus (as potassium phosphate) 2.75 mg/liter
Magnesium Aspartate 27.5 mg/liter
Zinc Monomethionine Aspartate 11 mcg/liter
Selenium (as amino-acid chelate) 5.5 mcg/liter
Copper Sebacate 13.75 mcg/liter
Manganese (as amino-acid chelate) 110 mg/liter
Chromium Picolinate 5.5 mcg/liter
Molybdenum (as amino-acid chelate) 2.75 mcg/liter
Vanadium Chelavite 2.75 mcg/liter
Sodium Chloride 60.5 mg/liter
Potassium Aspartate 121 mg/liter
L-Carnitine 300 mg/liter
Tyrosine 150 mg/liter
DL-Phenylalanine 150 mg/liter
CoEnzyme Q10 2.75 mg/liter
Choline Dihydrogen Citrate 27.5 mg/liter
OPC (Grape Seed Extract) 2.75 mg/liter
Inositol 1.1 mg/liter
PABA 0.55 mg/liter
Maltodextrin 5 g/liter
Sucralose 10 mg/liter
Acesulfame Potassium 20 mg/liter
Water balance

The foregoing example of nutraceutical beverage may be made without Sucralose, without Acesulfame Potassium or without both, as they are optional sweeteners. This nutraceutical beverage may be used if one requires a beverage with a wider range of nutritional ingredients.

1. A composition useful as an appetite satiation hydrating beverage, comprising about 200-400 mg/L hoodia gordonii, about 200-400 mg/L gymnema sylvestre, about 200-400 mg/L hydroxycitrate, about 250-500 mg/L green tea leaf extract, about 25-75 mg/L betaine, about 2-5 mg/L piperine, about 275-475 mg/L Potassium, about 50-100 mg/L Sodium, about 100-250 mg/L Vitamin C, about 5-25 g/L Maltodextrin.

2. The composition of claim 1, wherein the potassium is a chelated electrolyte in aspartate form.

3. The composition of claim 1, comprising the aspartate form of magnesium.

4. The composition of claim 1, comprising one or more of, vitamin E, selenium, coenzyme Q10, pycnogenol, L-glutathione, superoxide dismutase, bioflavonoids, pine bark grape seed complex, selenium, zinc, garlic or combinations thereof.

5. The composition of claim 4, comprising one or more of vitamin B1, vitamin B2, niacinamide, vitamin B6, vitamin B12, folic acid or combinations thereof.

6. The composition of claim 1, comprising one or more of calcium, zinc, vanadium, selenium, chromium, boron, manganese, copper or combinations thereof.

7. The composition of claim 1, comprising an amino acid.

8. The composition of claim 7, wherein the amino acid comprise one or more of, L-carnitine, lysine, isoleucine, leucine, threonine, valine, tryptophan, phenylalanine, methionine, L-selenomethionine, DL-phenylalanine, tyrosine or combinations thereof.

9. The composition of claim 1, comprising on or more of malic acid, citric acid or combinations thereof.

10. The composition of claim 1, comprising one or more of natural flavor, natural color, or combinations thereof.

11. The composition of claim 1, comprising a preservative.

12. The composition of claim 1 wherein said beverage is in carbonated.

13. A food product comprising at least one complex carbohydrate, at least one chelated electrolyte, Hoodia Gordonii Cactus, Gymnema Sylvestre, Betaine, and Piperine.

14. The composition of claim 13, comprising Hydroxycitrate.

15. The composition of claim 13, comprising Green Tea Leaf Extract.

16. The composition of claim 13, comprising Hydroxycitrate and Green Tea Leaf Extract

17. The composition of claim 16, wherein the concentration of Hoodia Gordonii Cactus is from about 80 mg/L to about 400 mg/L, Gymnema Sylvestre is from about 80 mg/L to about 400 mg/L, Green Tea Leaf Extract is about 100 mg/L to about 900 mg/L, Hydroxcitrate is about 80 mg/L to about 400 mg/L, Betaine is about 15 mg/L to about 100 mg/L, Piperine is about 1.1 mg/L to about 50 mg/L.

18. The composition of claim 13, comprising a nutritionally and appetite satiating effective amount of complex carbohydrates, of chelated electrolytes, of Hoodia Gordonii Cactus, of Gymnema Sylvestre, of Betaine, and of Piperine.

19. The composition of claim 13, wherein the chelated electrolytes, include chelate aspartate forms of potassium and magnesium.

20. The composition of claim 13, comprising an antioxidant.

21. The composition of claim 20, wherein the antioxidant comprises one or more of vitamin Ester-C, vitamin E, selenium, coenzyme Q10, pycnogenol, L-glutathione, superoxide dismutase, bioflavonoids, pine bark grape seed complex, selenium, zinc, garlic or combinations thereof.

22. The composition of claim 13, comprising sodium.

23. The composition of claim 22, wherein the sodium comprises one or more of sodium chloride, sodium citrate, sodium benzoate or combinations thereof.

24. The composition of claim 13, comprises one or more of the following sweetening agents, maltodextrin, neohesperidine dihydrochalcone, Lo Han Fruit extract, sucralose, or combinations thereof.

25. The composition of claim 24, wherein the level of sweeteners used is about 2% to 4% by weight of the beverage.

26. The composition of claim 13, wherein said Piperine comprises one or more of synthetically made Piperine extract from black pepper or extract from piper longum.

27. The composition of claim 13, comprising herbal extracts.

28. The composition of claim 27, wherein said herbal extracts comprises one or more of Curcumin, Boswellin, Ashwagandha, Ginkgo Biloba, Capsaicin, Aconitine or combinations thereof.

29. The composition of claim 13, further comprising vitamins, herbs, minerals and amino acids.

30. The composition of claim 29, wherein the vitamins comprise one or more of vitamin B1, vitamin B2, niacinamide, vitamin B6, vitamin B12, folic acid, vitamin C, vitamin E or combinations thereof.

31. The composition of claim 29, wherein the minerals comprise one or more of calcium, zinc, vanadium, selenium, chromium, boron, potassium, manganese, copper, magnesium or combinations thereof.

32. The composition of claim 29, wherein the amino acids comprise one or more of, L-carnitine, lysine, isoleucine, leucine, threonine, valine, tryptophan, phenylalanine, methionine, L-selenomethionine, DL-phenylalanine, tyrosine or combinations thereof.

33. The composition of claim 13, comprising malic acid and citric acid.

34. The composition of claim 13, comprising one or more of natural flavor, natural color, or combinations thereof.

35. The composition of claim 13, comprising a preservative.

36. The composition of claim 13, wherein said beverage is in carbonated.

37. The composition of claim 13, wherein said beverage is in a concentrate or powder form.

38. The composition of claim 13, solubilized in liquid and brought to a frozen state.

39. The composition of claim 13, wherein said composition is prepared as a compressed solid form.

40. The composition of claim 13, wherein the pH is below 4.6.

41. The composition of claim 13, wherein the pH is below 5.8.

42. A process for the manufacture of a liquid composition to be used as a appetite satiation and rehydration drink, wherein the components set forth in claim 13 are combined with sufficient liquid to provide a liquid composition ready for consumption by drinking.

43. A process for the manufacture of a liquid composition to be used as a appetite satiation and rehydration drink, wherein the components set forth in claim 13 are combined, the resulting combination being provided in an easy to open container ready for consumption by drinking.

44. A method of reducing one or more symptoms of appetite satiation and dehydration of a human body by administration to a subject of an effective amount of a composition as claimed in claim 13.

45. The use of at least one complex carbohydrate, at least one chelated electrolyte, Hoodia Gordonii Cactus, Gymnema Sylvestre, Betaine, and Piperine in a nutraceutical drink to suppress the appetite, increase fat metabolism, sensitize the glucostat in the brain that monitors blood sugar availability, hydrate and replenish electrolytes lost due to physical exertion and stress.
